(19) United States
(12) Patent Application Publication
(10) Pub. No.: US 2025/0312038 A1
(43) Pub. Date: Oct. 9, 2025

(54) SURGICAL STAPLER

(71) Applicant: NUtech Ventures, Lincoln, NE (US)

(72) Inventors: Mark A. Carlson, Lincoln, NE (US); Carl A. Nelson, Lincoln, NE (US); Nick Swerczek, Lincoln, NE (US); Peyton Kullmann, Lincoln, NE (US)

(21) Appl. No.: 19/081,883

(22) Filed: Mar. 17, 2025

Related U.S. Application Data
(60) Provisional application No. 63/575,465, filed on Apr. 5, 2024.

(51) Int. Cl. A61B 17/072; A61B 17/00

(52) U.S. Cl. CPC A61B 17/072; A61B 2017/00398; A61B 2017/07257; A61B 2017/07285

(57) ABSTRACT

A surgical instrument may include a tool assembly including a stapler assembly and an anvil assembly. The surgical instrument may include a handle assembly that includes a handle grip, a first cable drive assembly operatively coupled to the anvil assembly via a first cable, the first cable disposed within a channel assembly, a first trigger operatively coupled to the anvil assembly via the first cable, wherein an actuation of the first trigger causes the first cable to move from a protracted position to a retracted position, a second cable drive assembly operatively coupled to the stapler assembly via a second cable, the second cable disposed within the channel assembly; and a second trigger coupled to the stapler assembly via the second cable, wherein an actuation of the second trigger causes the stapler assembly to drive staples into a tissue.

### CROSS-REFERENCE TO RELATED APPLICATION

The present application claims the benefit under 35 U.S.C. § 119(e) of U.S. Provisional Application Ser. No. 63/575,465, filed Apr. 5, 2024, which is incorporated herein by reference in the entirety.

### TECHNICAL FIELD

The present invention generally relates to surgical applications and, in particular, to surgical staplers.

### BACKGROUND

Surgical staplers are commonly used for closing surgical wounds. Several sizes and designs of surgical staplers have been developed based on specific surgical needs. For colectomies, a procedure for treating colon and rectal diseases and obstructions, surgical staplers are used for transecting the colon, which are often performed laparoscopically, requiring the introduction of the stapler, or stapler head, through a small incision into the abdominal cavity, typically through a laparoscopic trocar. The small incision and the confined spaces of the abdominal cavity can make it difficult for the surgeon to insert and properly position the head of the surgical stapler (which can have a head articulation range of 45-60 degrees) relative to a proper site and angle of transection (e.g., an angle approximately perpendicular to a length of the colon), potentially causing an unfavorable outcome.

Current surgical staplers used in colectomies typically include flexible pushrods that are used to actuate both the articulation of the stapler head and for stapling and/or cutting action. The incorporation of the pushrods constrains the ability of the stapler head to articulate when deployed. As a result, the stapler head can only articulate approximately 45-60 degrees from a straight configuration, making it difficult to staple and/or cut the tissue at the proper angle, such as the desired right angles relative to the long axis of the tissue or organ (e.g., most commonly but not limited to the lower rectum).

Therefore, it would be advantageous to provide systems and methods that overcome the shortcomings described above.

### SUMMARY

A surgical instrument is disclosed, in accordance with one or more embodiments of the present disclosure. In embodiments, the surgical instrument includes a tool assembly. In embodiments, the tool assembly includes a stapler assembly and an anvil assembly pivotably coupled to the stapler assembly and configured to move between a spaced position and a clamped position relative to the stapler assembly in response to a movement of a first cable from a protracted position to a retracted position. In embodiments, the surgical instrument includes a channel assembly coupled to the tool assembly on a distal end, wherein the channel assembly and the tool assembly are configured to articulate at an articulation joint. In embodiments, the surgical instrument includes a handle assembly coupled to the channel assembly on a proximal end, the handle assembly including: a handle grip; a first cable drive assembly operatively coupled to the anvil assembly via a first cable, the first cable disposed within the channel assembly; a first trigger operatively coupled to the anvil assembly via the first cable, wherein an actuation of the first trigger causes the first cable to move from the protracted position to the retracted position; a second cable drive assembly operatively coupled to the stapler assembly via a second cable, the second cable disposed within the channel assembly; and a second trigger coupled to the stapler assembly via the second cable, wherein an actuation of the second trigger causes the stapler assembly to drive staples into a tissue.

In one or more embodiments, the handle assembly further includes an articulation control element; and an articulation cable drive assembly coupled to the articulation control element and the tool assembly via an articulation cable.

In one or more embodiments, the first cable drive assembly further includes a first gear spool including a first gear component configured to rotate upon an input from the first trigger; and a first spool component operatively coupled to the first cable, wherein the first spool component is configured to move the first cable upon a rotation by the first gear component.

In one or more embodiments, the surgical instrument further includes a locking assembly including a locking lever, the locking lever including: a pawl disposed within a housing of the handle assembly and biased against the first gear spool; and a press point extending outside of the housing, wherein pressing the press point biases the pawl away from the first gear spool.

In one or more embodiments, the second cable drive assembly includes a second gear spool including a second gear component configured to rotate upon an input from the second trigger; and a second spool component operatively coupled to the second cable, wherein the second spool component is configured to move the second cable upon a rotation by the second gear component.

In one or more embodiments, the first cable drive assembly and the second cable drive assembly are operatively coupled, wherein a triggering of the first trigger causes the second trigger to articulate to a triggerable position.

In one or more embodiments, the surgical instrument, further includes a first four-bar linkage coupled to the first trigger, including a first gear wedge configured to bias a first gear spool when the first trigger is pulled; and an interlock pin disposed on the first gear wedge; and a second four-bar linkage coupled to the second trigger, including: a second gear wedge configured to bias a second gear spool when the second trigger is pulled; and an interlock slot disposed on the second gear wedge configured to receive the interlock pin.

In one or more embodiments, the first trigger includes an inverted handle that pivots at a fulcrum.

In one or more embodiments, the surgical instrument further includes a pin interlock, wherein the pin interlock prevents the second trigger from being pulled until the first trigger is pulled.

In one or more embodiments, the handle assembly is configured to trigger the tool assembly while the tool assembly is inserted into a subject via a trocar insertion site.

In one or more embodiments, the surgical instrument is configured to staple tissues within a subject during a laparoscopic procedure.

Another surgical instrument is disclosed, in accordance with one or more embodiments of the present disclosure. In embodiments, the surgical instrument includes: a tool assembly; a channel assembly coupled to the tool assembly on a distal end via an articulation joint; a handle assembly coupled to the channel assembly on a proximal end, the handle assembly including: a handle grip; an articulation element; and an articulation cable drive assembly coupled to the articulation element and the tool assembly via an articulation cable, wherein an adjustment of the articulation element causes the tool assembly to articulate greater than 60 degrees relative to the channel assembly.

In one or more embodiments, the adjustment of the articulation element causes the tool assembly to articulate approximately 90 degrees relative to the channel assembly.

In one or more embodiments, the articulation joint includes a revolute articulation joint.

In one or more embodiments, the surgical instrument includes a single revolute articulation joint.

In one or more embodiments, the surgical instrument, further includes a first front idler pulley disposed within the articulation joint and a first rear idler pulley disposed within the tool assembly, wherein a first cable is threaded through the first front idler pulley and the first rear idler pulley.

In one or more embodiments, the surgical instrument, further includes a second front idler pulley disposed within the articulation joint and a second rear idler pulley disposed within the tool assembly, wherein a second cable is threaded through the second front idler pulley and the second rear idler pulley.

In one or more embodiments, the handle assembly is configured to trigger the tool assembly while the tool assembly is inserted into a subject via a trocar insertion site.

In one or more embodiments, the surgical instrument is configured to staple tissues within a subject during a laparoscopic procedure.

In embodiments, a method for operating a surgical instrument is disclosed. In one or more embodiments, the method includes obtaining the surgical instrument, wherein the surgical instrument includes: a tool assembly including an anvil assembly and a stapler assembly; a channel assembly coupled to the tool assembly on a distal end; and a handle assembly coupled to the channel assembly on a proximal end including a first trigger and a second trigger. In one or more embodiments, the method includes inserting the tool assembly into a body cavity while the anvil assembly is in a clamped position relative to the stapler assembly. In one or more embodiments, the method includes opening the anvil assembly to an open position relative to the stapler assembly. In one or more embodiments, the method includes positioning tissue between the anvil assembly and the stapler assembly; pulling a first trigger, wherein pulling the first trigger moves the anvil assembly into a clamped position, and moves the second trigger into a triggerable position. In one or more embodiments, the method includes pulling a second trigger, wherein pulling the second trigger causes the stapler assembly to drive staples into the tissue.

### BRIEF DESCRIPTION OF THE DRAWINGS

The detailed description is described with reference to the accompanying figures. The use of the same reference numbers in different instances in the description and the figures may indicate similar or identical items. Various embodiments or examples (“examples”) of the present disclosure are disclosed in the following detailed description and the accompanying drawings. The drawings are not necessarily to scale. In general, operations of disclosed processes may be performed in an arbitrary order, unless otherwise provided in the claims.

FIG. 1 illustrates a side view of a surgical stapler with a portion of the housing removed, in accordance with one or more embodiments of this disclosure.

FIG. 2A illustrates a perspective view of the tool assembly in a closed position, in accordance with one or more embodiments of this disclosure.

FIG. 2B illustrates an exploded perspective view of the tool assembly in a closed position, in accordance with one or more embodiments of this disclosure.

FIG. 2C illustrates an exploded side-view of the tool assembly, in accordance with one or more embodiments of the disclosure.

FIG. 2D illustrates a detailed perspective view of the tool assembly in a closed position, in accordance with one or more embodiments of this disclosure.

FIG. 3 illustrates a semi-transparent side-view of the articulation joint, in accordance with one or more embodiments of the disclosure.

FIG. 4 illustrates a cross-section view of the handle assembly, in accordance with one or more embodiments of the disclosure.

FIG. 5 is a flow diagram illustrating steps performed in a method for operating a surgical instrument in accordance with one or more embodiments of the disclosure.

### DETAILED DESCRIPTION

The present disclosure has been particularly shown and described with respect to certain embodiments and specific features thereof. The embodiments set forth herein are taken to be illustrative rather than limiting. It should be readily apparent to those of ordinary skill in the art that various changes and modifications in form and detail may be made without departing from the spirit and scope of the disclosure.

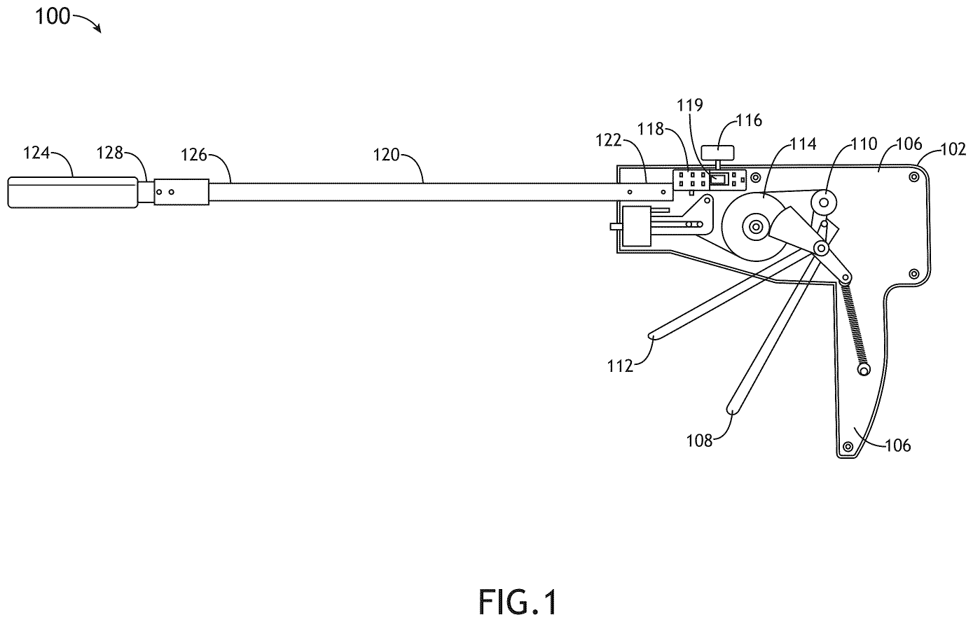

FIGS. 1-5 generally illustrate a surgical stapler in accordance with one or more embodiments of the present disclosure.

Embodiments of the present disclosure are directed to a surgical stapler, a surgical instrument used in colectomies and other types of surgeries that require staples to be placed inside a body cavity. The surgical stapler may include a tool assembly for clamping and stapling tissue within the body of a patient, a hand assembly for operating the tool assembly from outside the body of a patient, and a long channel assembly that connects to the tool assembly and the hand assembly. The channel assembly and the tool assembly are coupled via an articulation joint, such as a revolute joint that enables the tool assembly to pivot more than 60 degrees, and up to or more than 90 degrees relative to the channel assembly. The articulation joint assists the medical practitioner in stapling organs and tissues at right angles relative to the length of the tissue or organ. The hand assembly controls the articulation of the tool assembly, as well as the clamping and stapling actions of the tool assembly, via a series of cables that are threaded through the channel assembly and the articulation joint. The increased flexibility of the assembly tool facilitates the proper positioning of the assembly tool to the stapling target. Due to the small diameter of the tool assembly and the flexibility of the articulation joint, the surgical stapler can be used in minimally invasive laparoscopic procedures on tissues and organs deep within the abdominal and pelvic regions.

FIG. 1 illustrates a side view of a surgical stapler 100 with a portion of the housing 102 removed, in accordance with one or more embodiments of this disclosure. The surgical stapler 100 may be used for resection (e.g., removing a part of a tissue or organ), transection (e.g., cutting through and sealing organs and tissues), and creating connections (e.g., anastomoses). The surgical stapler 100 may be used in any surgery requiring tissue stapling including, but not limited to, colectomy, proctocolectomy, lleoanal anastomosis hemicolectomy, duodenectomy, jejunectomy, ileectomy, Whipple procedures, and/or other endoscopic and laparoscopic surgeries. The surgical stapler may include, or be integrated within, any type of surgical instrument required to staple tissues within a patient, such as surgical instruments used during surgeries where at least part of the instrument is inserted through an incision (e.g., hole, port, or trocar insertion site). These surgical instruments may include, but not be limited to, an endoscope, a laparoscope, a gastroscope, a cystoscope, a colonoscope, an arthroscope, a urethroscope, a hysteroscope, a rhinoscope, a laryngoscope, an endoscopic stapler and cutter, a stapling and tissue sealing device, trocars, laparoscopic scissors, laparoscopic graspers, laparoscopic forceps, electrocautery instruments, suction devices, endoscopic scalpels, endoscopic retractor, endoscopic snares, laparoscopic clips, and laparoscopic fasteners.

In embodiments, the surgical stapler 100 includes a handle assembly 104 that includes the housing 102 and a handle grip 106. The handle assembly 104 may further include a first trigger 108 coupled to a first cable drive assembly 110 and a second trigger 112 coupled to a second cable drive assembly 114. The handle assembly 104 may further include an articulation control element 116 (e.g., a knob) operatively coupled to an articulation cable drive 118 that includes a drive gear 119.

In embodiments, the surgical stapler 100 includes a channel assembly 120 (e.g., a hollow shaft) that is attached to the handle assembly 104 on a proximal end 122 (e.g., the end closest to the operator). The surgical stapler may further include a tool assembly 124 coupled to the channel assembly on a distal end 126 via an articulation joint 128. The articulation joint 128 facilitates movement of the tool assembly relative to the channel assembly 120. Movement and activation (e.g., cutting and stapling) of the tool assembly 124 are controlled manually by control elements in the handle assembly 104 (e.g., the first trigger 108, the second trigger 112, and the articulation control element 116). The control elements are coupled to their respective component within the tool assembly 124 via a set of cables that are routed within the hollow channel assembly 120 and articulation joint 128 to the tool assembly 124.

The tool assembly 124 includes one or more tools used for surgery including, but not limited to, a clamp, a stapler, and a knife. For example, the tool assembly 124 may include components that can clamp tissue, such as a section of the colon. In another example, the tool assembly 124 may include components that can staple tissues together, such as the stapling required to seal a section of the colon. In another example, the tool assembly 124 may include components that cut tissue, such a knife that can cut off a section of colon after one or both sides of the colon section has been stapled. For instance, the tool assembly 124 may include componentry for sequentially clamping the colon section and stapling the colon section. In another instance, the tool assembly 124 may include componentry for sequentially clamping the colon section, stapling the colon section, and cutting the colon section between two or more lines of staples.

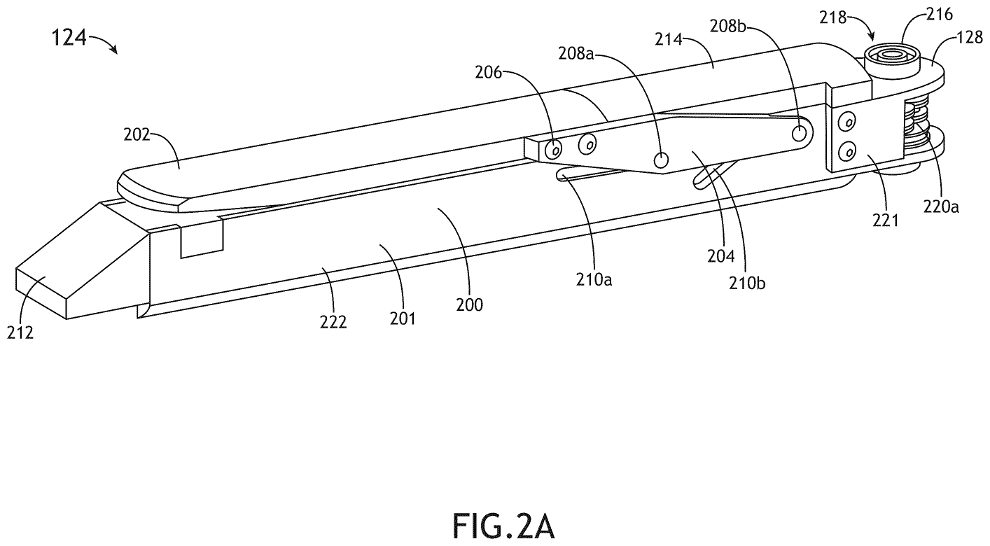

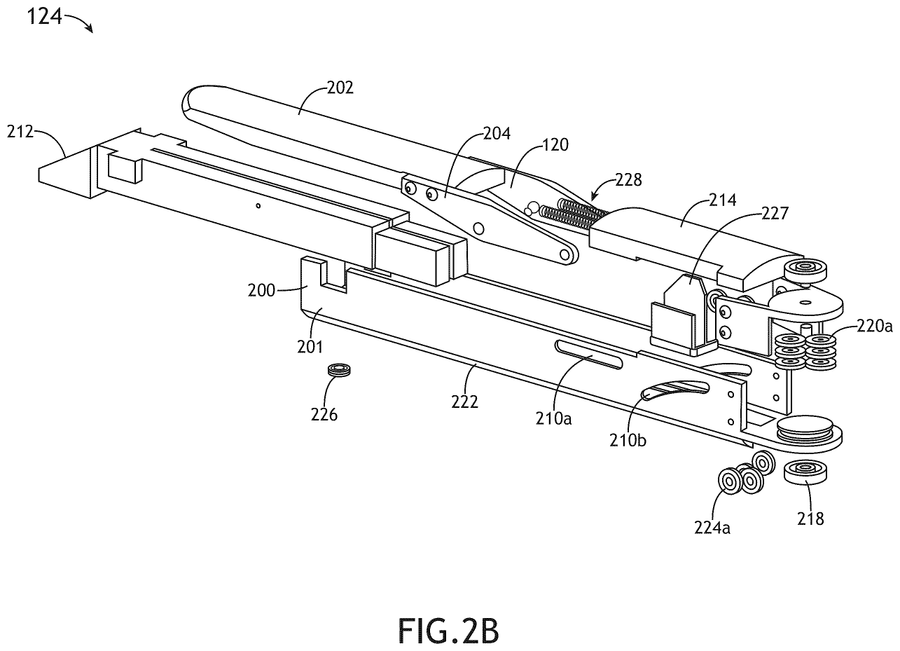

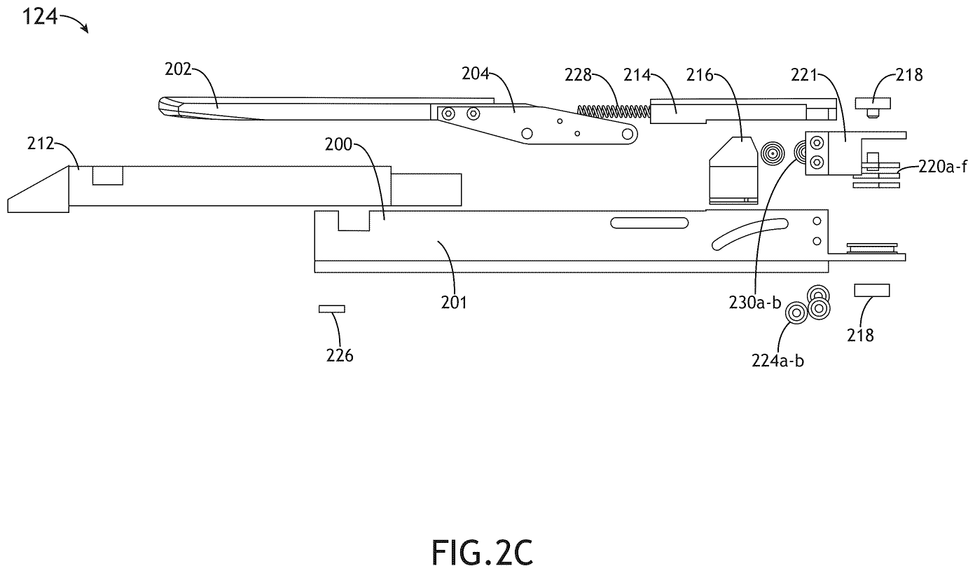

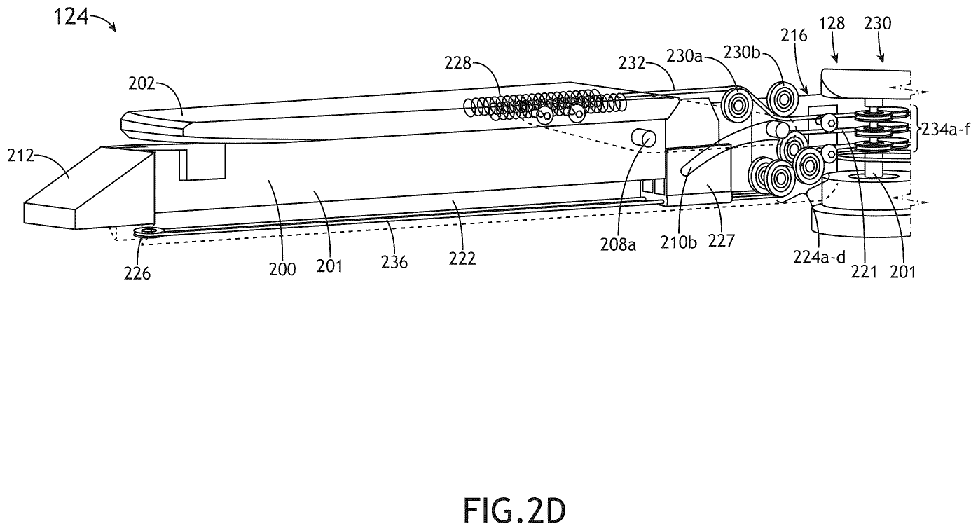

FIG. 2A illustrates a perspective view of the tool assembly 124 in a closed position, in accordance with one or more embodiments of this disclosure.

In embodiments, the tool assembly 124 includes a stapler assembly 200 (e.g., lower jaw with stapler housing 201), and an anvil assembly 202 (e.g., upper jaw). The stapler assembly 200 and the anvil assembly 202 are pivotably coupled via a guide flange 204. The guide flange 204 is coupled to the anvil assembly 202 via connectors 206 (e.g., rivets or screws) and is slidably coupled to the stapler assembly 200. For example, the guide flange 204 may include one or more cams 208a-b that slidably couple to one or more cam slots 210a-b integrated into the stapler housing 201. The stapler assembly 200 may include a removable staple cartridge 212 comprising one or more sets of staples that are positioned for stapling by the stapler assembly 200. The stapler assembly 200 may include one or more dead space caps 214 to prevent bodily fluid and tissues from entering dead spaces within the tool assembly 124. FIG. 2A further illustrates a tool portion 216 of the articulation joint 128 containing one or more articulation bearings 218 and a set of front idler pulleys 220a-f for controlling cable tension within the articulation joint 128. The tool portion 216 is coupled to the stapler assembly 200 via a joint cross brace 221. The stapler assembly 200 may further include an underjaw area 222 below the seating space of the staple cartridge 212.

FIG. 2B illustrates an exploded perspective view of the tool assembly 124 in a closed position, in accordance with one or more embodiments of this disclosure.

In embodiments, the tool assembly 124 includes rear idler pulleys 224a-d that, along with the front idler pulleys, 220a-f guide cables that cross over between the tool assembly 124 and the channel assembly 120. The tool assembly 124 may further include a distal pulley 226 for routing cables involved in cutting and/or stapling actions. In embodiments, the tool assembly 124 includes a knife carriage 227 that translates length-wise across the stapler assembly 200 to cut tissue after the tissue has been stapled. The knife carriage 227 may be configured to secure a knife or may itself include a cutting surface.

FIG. 2C illustrates an exploded side-view of the tool assembly 124, in accordance with one or more embodiments of the disclosure.

In embodiments, the anvil assembly 202 includes return springs 228 (e.g., compressive springs) that bias against the anvil assembly 202 and/or the guide flange 204. The biasing of the return springs causes a movement of the anvil assembly that is guided by the cams 208a-b sliding along the guide slots 210a-b. In particular, guide slot 210b has a sloping line that slopes downward toward the distal end of the tool assembly 124. Because of the downward slope, the movement caused by the biasing of the return springs 228 causes the anvil assembly to separate or open up from the stapler assembly 200 into an open or spaced position. FIG. 2C further illustrates a set of anvil idler pulleys 230a-b for guiding cables that control the operation of the anvil assembly 202.

FIG. 2D illustrates a semi-transparent perspective side view of the tool assembly 124 and the articulation joint, in accordance with one or more embodiments of the disclosure. The semi-transparent components of the side-view permit viewing of cabling within the tool assembly 124 and the articulation joint 128.

In embodiments, the surgical stapler 100 includes a first cable 232 that is threaded through the anvil idler pulleys 230a-b, a set of front idler pulleys 234a-b, and is coupled to the anvil assembly. When the first cable 232 is pulled (e.g., from a protracted position to a retracted position), the anvil assembly 202 responds by articulating from the open or spaced position to a clamped position due to the sliding of the cams 208a-b of the guide flange 204 along the cam slots 210a-b (e.g., the anvil assembly 202 is configured to move between a spaced position and a clamped position relative to the stapler assembly 200 in response to a movement of a first cable 232 from a protracted position to a retracted position). When tension in the first cable 232 is released, the return springs bias the anvil assembly 202 back to the open position.

In embodiments, the surgical stapler includes a second cable 235 that is threaded through a set of front idler pulleys 234c-d and is coupled to the stapler assembly 200. For example, when the second cable 235 is pulled, the stapler assembly 120 is actuated, causing a set of staples to be driven into tissue that is clamped between the anvil assembly 202 and the stapler assembly 200. Stapling mechanisms in surgical staplers are well-known to the skilled artisan and are not the focus of this disclosure.

In embodiments, the surgical stapler 100 includes a knife cable 236 that is coupled to the knife carriage 227. The knife cable 240 is threaded through a set of front idler pulleys 234e-f and a set of rear idler pulleys 224a-b. The knife cable 240 is further threaded or routed through the underjaw area 222 to the distal pulley 226. When the knife cable 240 is pulled, the knife carriage 227 travels along the length of the underjaw area 222 (from approximately the rear idler pulleys 22a-b to the distal pulley 226), cutting tissue that has been clamped and stapled by the tool assembly 124. In embodiments, mechanisms for movement of the knife carriage and actuation of the stapler mechanism of the stapler assembly are coupled. For example, a cutting action associated with the knife carriage and the actuation of the stapler assembly may be facilitated by a single cable (e.g., the knife cable or second cable) or a single cable set. For instance, the knife carriage 227 may include staple actuation hardware, such as a staple driver that forces staples into the tissue before the knife carriage 227 travels along the underjaw area 222 to cut tissue adjacent to the stapled tissue.

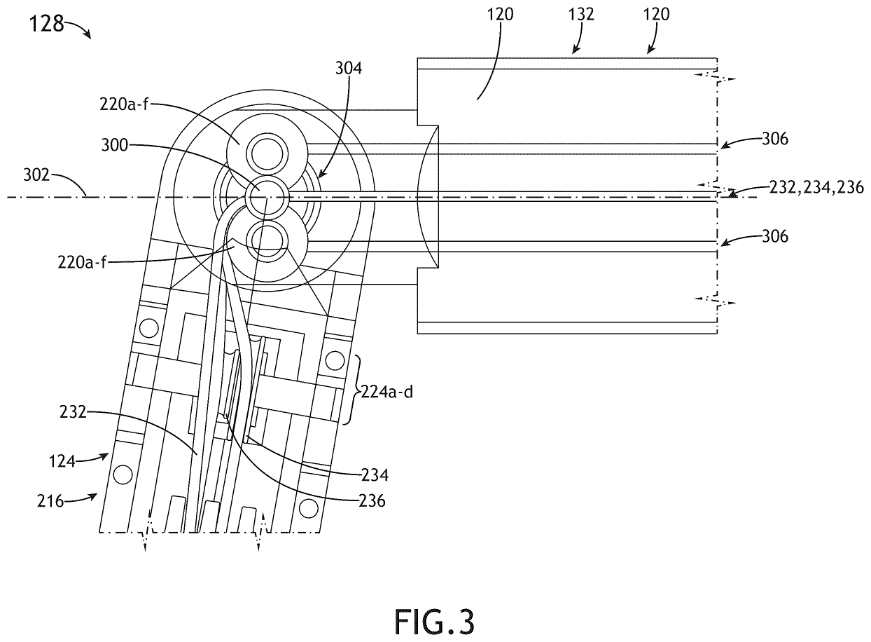

FIG. 3 illustrates a semi-transparent side-view of the articulation joint 128, in accordance with one or more embodiments of the disclosure.

In embodiments, the articulation joint 128 includes an articulation axis 300 about which the channel assembly 120 and the tool assembly 124 pivot relative to each other (e.g., the tool assembly pivots relative to the channel axis 302 of the channel assembly 120. Pairs of front idler pulleys 220a-f are positioned around the articulation axis 300 so that the articulation axis 300 is located between each front idler pulley pair. Cables (e.g., the first cable 232, second cable 235, knife cable 240) that are threaded through the front idler pulleys 220a-f are then also threaded through, or close to, the articulation axis 300. This arrangement of cables saves space (e.g., reduced dead space) and facilitates a greater articulation range of the tool assembly 124 relative to the channel assembly 120 and channel axis 302 than earlier designs, such as surgical staplers using continuum flexure joints.

The articulation joint 128 may include any type of joining structure including, but not limited to, a revolute joint. Revolute joints are one-degree-of-freedom joints that allow pivoting with large articulation ranges. In particular, the position of the cables 232, 235, 240 inside the revolute articulation joint 128 at or near the articulation axis 300 allows the articulation joint 128 to articulate to a greater extent than articulation joints of other surgical staplers that flexure joints or pushrods instead of cables. For example, articulated surgical staplers that use flexure joints and/or pushrods and are typically limited to an articulation range of 45° relative to the channel axis 302 (e.g., the movement of the tool assembly 124 from a straight or unbent position relative to the channel axis 302 or channel assembly 120, as shown in FIG. 1, to the furthest limit of articulation between the tool assembly 124 and the channel assembly 120). The use of a revolute articulation joint 128 reduces both the complexity and housing and/or dead spaces of the componentry required for other articulation joints not using the revolute joint structure.

In embodiments, the tool assembly 124 is configured to articulate more than 60 degrees (e.g., from the straight position to the furthest articulation position), more than 70 degrees, more than 80 degrees, or more than 90 degrees. For example, the tool assembly 124 may be configured to articulate approximately 90 degrees from a straight position to the furthest articulation position relative to the channel assembly 120, without negatively affecting the functions of the surgical stapler (e.g., clamping, stapling, and cutting).

In embodiments, the surgical stapler 100 includes an articulation gear 304 and an articulation cable 306 threaded around the articulation gear. When the articulation cable 306 is pulled, the articulation gear 304 rotates, resulting in the tool assembly 104 rotating relative to the channel assembly 120. The articulation cable 306 is also threaded around the drive gear 119 of the articulation cable drive 118 that is controlled by the articulation control element 116 (e.g., knob) as shown in FIG. 1. A manual adjustment or twisting of the knob causes the drive gear to rotate, which causes a corresponding rotation of the articulation gear 304 via the articulation cable 306.

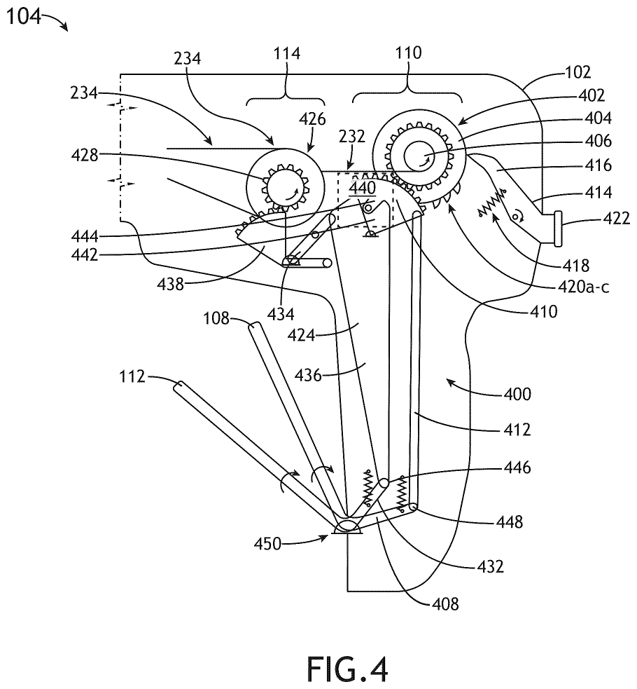

FIG. 4 illustrates a cross-section view of the handle assembly 104, in accordance with one or more embodiments of the disclosure.

In embodiments, the first cable drive assembly 110 includes a first four-bar linkage 400 coupled to the first trigger 108 and a first gear spool 402 that is coupled to the first cable 232. The first gear spool 402 may include a first gear component 404 and a first spool component 406 that wraps the first cable 232 onto the first gear spool 402 when the gear spool 402 is rotated. The first four-bar linkage 400 may include a first input link 408 coupled to the first trigger 108, a first output link (e.g., first gear wedge 410), and a first coupler link 412 that couples the first input link 408 to the first gear wedge 410. The first four-bar linkage 400 and the first gear spool 402 are operatively coupled. For example, a pulling of the first trigger 108 results in the movement of teeth of the first gear wedge 410 that biases the teeth of the first gear spool 402 (e.g., the first gear wedge 410 meshes with the first gear spool 402). This biasing causes the first gear spool to rotate, which spools or advances the first cable 232 onto the first spool component 406.

In embodiments, the handle assembly 104 may include a locking lever 414 that is disposed at least partially within the housing 102. The locking lever 414 may include a pawl 416 that is biased against the first gear spool 402 (e.g., via a pawl spring 418). When the first gear spool 402 is rotated, the pawl 416 locks the first gear spool 402 via one or more ratchet teeth 420a-c and prevents the first cable 232 from spooling (e.g., the action of the first trigger 108, the first gear spool 402 and locking lever 414 acting as a ratchet) The locking lever may include a press point 422 (e.g., a press point 422 extending outside of the housing 102) that when pressed biases the pawl 416 away from the first gear spool 402, and allowing the first cable 232 to unwind if the first trigger 108 is no longer pressed.

In embodiments, the second cable drive assembly 114 includes a second four-bar linkage 424 coupled to the second trigger 112 and a second gear spool 426. The second gear spool 426 may include a second gear component 428 and a second spool component 430 that wraps the second cable 235 onto the second gear spool 426 when the second gear spool 426 is rotated. The second four-bar linkage 424 may include a second input link 432 coupled to the second trigger 112, a second output link 434, and a second coupler link 436 that couples the first input link 408 to the first gear wedge 410. The second four-base link may further include a second gear wedge 438 coupled to the second output link 434. The second four-bar linkage 424 and the second gear spool 426 are operatively coupled. For example, a pulling of the second trigger 112 results in the movement of teeth of the second gear wedge 438 that biases the teeth of the second gear spool 426 (e.g., the second gear wedge 438 meshes with the first gear spool 402). This biasing causes the second gear spool 426 to rotate, which spools the second cable 235 onto the second spool component 430.

In embodiments, the first cable drive assembly 110 and the second cable drive assembly 114 are operatively coupled via a pin interlock 440. The pin interlock 440 may include an interlock pin 442 (e.g., disposed on the first gear wedge 410) received by, and configured to slide along, an interlock slot 444 (e.g., integrated into the second coupler link 436). When the first trigger 108 is fully pulled or depressed, the interlock pin 442 guides the movement of the second cable drive assembly 114, resulting in several motions that place the second trigger 112 in a triggerable position. First, when the first trigger 108 is pulled, the motion of the interlock pin 442 and the interlock slot 444 will cause the second trigger 112 to rotate (e.g., approximately 30 degrees) toward the vertical (e.g., into a triggerable position), as well as cause the anvil assembly 202 to move to a clamped position via the first cable 232. This rotation will make it easier for the operator to reach the second trigger 112. Second, the motion will cause the second output link 434 to be moved to a firing position. Third, upon reaching the fully pulled position, the interlock pin 442 will be freed from the interlock slot 444, allowing the second trigger 112 to be pulled.

In embodiments, the handle assembly 104 may include a first trigger 108 and/or a second trigger 112 that is inverted. For example, the first trigger and/or second trigger 112 may include a respective first trigger joint 446 and a second trigger joint 448 that enable both the first trigger 108 and the second trigger 112 to be designed with an inverted orientation that pivots at a fulcrum 450 so that the respective first coupler link 412 and second coupler link 436 are pulled downward, engaging the respective first gear wedge 410 and second gear wedge 438.

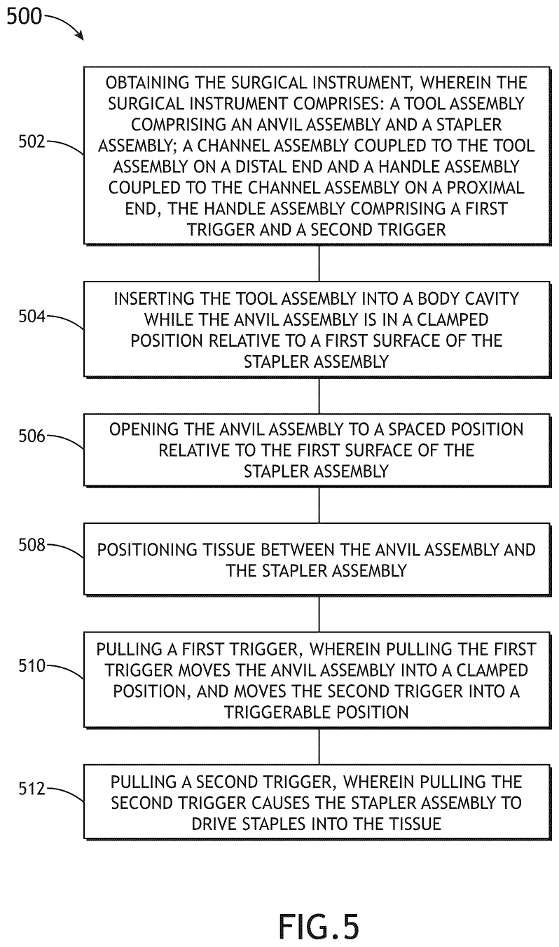

FIG. 5 is a flow diagram illustrating steps performed in a method 500 for operating a surgical instrument (e.g., the surgical stapler 100) in accordance with one or more embodiments of the disclosure. The method 500 may be performed during resection, transection, and anastomose surgeries as described herein.

In embodiments, the method 500 includes a step 502 of obtaining the surgical instrument, wherein the surgical instrument (e.g. the surgical stapler 100) comprises a tool assembly 124 comprising an anvil assembly 202 and a stapler assembly 200, a channel assembly 120 coupled to the tool assembly on a distal end 126, and a handle assembly 104 coupled to the channel assembly 120 on a proximal end 122 comprising a first trigger 108 and a second trigger 112. The surgical stapler may also include other components (e.g., the knife carriage 227 or the locking lever 414) as described herein.

In embodiments, the method 500 includes a step 504 of inserting the tool assembly 124 into a body cavity while the anvil assembly 202 is in a clamped position relative to the stapler assembly 200. For example, during a laparoscopic surgery, where a small hole (e.g., 1-4 cm) is produced at the abdominal or pelvic region of the patient, the tool assembly 124 is introduced through the small hole in the most compact position possible, which is the clamped position. The anvil assembly 202 is biased into the clamped position via the return springs 228.

In embodiments, the method 500 includes a step 506 of opening the anvil assembly to a spaced position relative to the stapler assembly. Forces for opening the anvil assembly 202 may include the biasing force provided by the return springs 228. Opening the anvil assembly 202 may also include a release of tension provided by the first cable 232 and the first trigger 108. For example, the first trigger may be positioned or moved so that the tension of the first cable 232 is released, enabling the return springs to be moved to the spaced position.

In embodiments, the method 500 includes a step 508 of positioning tissue between the anvil assembly 202 and the stapler assembly 200. For example, the operator may cause an articulation at the articulation joint 128 (e.g., via the articulation control element 116) that moves the tool assembly 124 into a position that places the tissue or organ between the anvil assembly 202 and the stapler assembly 200 (e.g., at a position approximately perpendicular to a length of the tissue or organ). In another example, the entire tool assembly may be repositioned. In another example, the tissue or organ is placed between the anvil assembly 202 and the stapler assembly 200 via another surgical tool, such as forceps or hemostats. The positioning of the tissue or organ between the anvil assembly 202 and the stapler assembly 200 may involve a combination of these steps.

In embodiments, the method 500 includes a step 510 of pulling a first trigger 108, wherein pulling the first trigger 108 moves the anvil assembly 202 into a clamped position, and moves the second trigger 112 into a triggerable position. For example, pulling the first trigger 108 causes the first gear spool 402 to rotate, which causes the interlock pin 442 to slide along the interlock slot 444. This causes the first coupler link 412 to translate, which then causes the first input link 408 and the first gear wedge 410 to rotate clockwise, which moves the second trigger to the triggerable position.

In embodiments, the method 500 includes a step 512 of pulling a second trigger 112, wherein pulling the second trigger 112 causes the stapler assembly 200 to drive staples into the tissue. For example, the stapler assembly 200 may drive a single line of staples into the tissue, creating an anastomosis. In another example, the stapler assembly may drive two lines of staples into the tissue in preparation for a cutting action between the two lines that will separate the two tissues. The tool assembly 124 may then be removed from the body cavity.

In embodiments, the method 500 includes a step of articulating the tool assembly 124 greater than 60 degrees relative to the channel assembly 120 (e.g., between the steps of inserting the tool assembly 124 and opening the anvil assembly 202). For example, the medical practitioner may manually operate the articulation cable drive 118 by twisting the articulation control element 116 (e.g., knob), causing the tool assembly 124 that has been inserted within the body cavity to rotate to a position 60 degrees or more relative to the channel assembly 120. The tool assembly may be rotated further as described herein.

In embodiments, the method 500 may include a step of cutting the tissue via the knife carriage 227. For example, the medical practitioner may operate a trigger or lever of a knife cable drive assembly operatively or other drive assembly operatively coupled to the knife carriage 227 via the knife cable, causing the knife carriage 227 to traverse the length of the tool assembly 124, cutting the tissue. The step of cutting the tissue may occur at the conclusion of the stapling step 512 and may be a consequence of the stapling step 512 (e.g., the cutting step is initiated at or by the conclusion of the stapling step 512).

In embodiments, the surgical stapler 100 is configured to staple tissues within a subject during a laparoscopic procedure. For example, the handle assembly 104 may be configured to trigger the tool assembly 124 while the tool assembly 124 is inserted into a subject (e.g., a patient) via a trocar insertion site.

As used throughout, “at least one” means one or a plurality of; for example, “at least one” may comprise one, two, three, . . . , one hundred, or more. Similarly, as used throughout, “one or more” means one or a plurality of; for example, “one or more” may comprise one, two, three, . . . , one hundred, or more. Further, as used throughout, “zero or more” means zero, one, or a plurality of; for example, “zero or more” may comprise zero, one, two, three, . . . , one hundred, or more.

In the present disclosure, the methods, operations, and/or functionality disclosed may be implemented as sets of instructions or software readable by a device. Further, it is understood that the specific order or hierarchy of steps in the methods, operations, and/or functionality disclosed are examples of exemplary approaches. Based upon design preferences, it is understood that the specific order or hierarchy of steps in the methods, operations, and/or functionality can be rearranged while remaining within the scope of the inventive concepts disclosed herein. The accompanying claims may present elements of the various steps in a sample order and are not necessarily meant to be limited to the specific order or hierarchy presented.

It is to be understood that embodiments of the methods disclosed herein may include one or more of the steps described herein. Further, such steps may be carried out in any desired order and two or more of the steps may be carried out simultaneously with one another. Two or more of the steps disclosed herein may be combined in a single step, and in another embodiment, one or more of the steps may be carried out as two or more sub-steps. Further, other steps or sub-steps may be carried in addition to, or as substitutes to one or more of the steps disclosed herein.

Before explaining at least one embodiment of the inventive concepts disclosed herein in detail, it is to be understood that the inventive concepts are not limited in their application to the details of construction and the arrangement of the components or steps or methodologies set forth in the following description or illustrated in the drawings. In the following detailed description of embodiments of the instant inventive concepts, numerous specific details are set forth in order to provide a more thorough understanding of the inventive concepts. However, it will be apparent to one of ordinary skill in the art having the benefit of the instant disclosure that the inventive concepts disclosed herein may be practiced without these specific details. In other instances, well-known features may not be described in detail to avoid unnecessarily complicating the instant disclosure. The inventive concepts disclosed herein are capable of other embodiments or of being practiced or carried out in various ways. Also, it is to be understood that the phraseology and terminology employed herein are for the purpose of description and should not be regarded as limiting.

As used herein a letter following a reference numeral is intended to reference an embodiment of the feature or element that may be similar, but not necessarily identical, to a previously described element or feature bearing the same reference numeral (e.g., 1, 1a, 1b). Such shorthand notations are used for purposes of convenience only and should not be construed to limit the inventive concepts disclosed herein in any way unless expressly stated to the contrary.

Further, unless expressly stated to the contrary, “or” refers to an inclusive or and not to an exclusive or. For example, a condition A or B is satisfied by anyone of the following: A is true (or present), and B is false (or not present), A is false (or not present), and B is true (or present), and both A and B are true (or present).

In addition, use of the “a” or “an” are employed to describe elements and components of embodiments of the instant inventive concepts. This is done merely for convenience and to give a general sense of the inventive concepts, and “a” and “an” are intended to include one or at least one and the singular also includes the plural unless it is obvious that it is meant otherwise.

Finally, as used herein, any reference to “one embodiment,” or “some embodiments” means that a particular element, feature, structure, or characteristic described in connection with the embodiment is included in at least one embodiment of the inventive concepts disclosed herein. The appearances of the phrase “in some embodiments” in various places in the specification are not necessarily all referring to the same embodiment, and embodiments of the inventive concepts disclosed may include one or more of the features expressly described or inherently present herein, or any combination of sub-combination of two or more such features, along with any other features which may not necessarily be expressly described or inherently present in the instant disclosure.

What is claimed:

1. A surgical instrument comprising:
a tool assembly comprising:
a stapler assembly; and
an anvil assembly pivotably coupled to the stapler assembly and configured to move between a spaced position and a clamped position relative to the stapler assembly in response to a movement of a first cable from a protracted position to a retracted position;
a channel assembly coupled to the tool assembly on a distal end, wherein the channel assembly and the tool assembly are configured to articulate at an articulation joint; and
a handle assembly coupled to the channel assembly on a proximal end, the handle assembly comprising:
a handle grip;
a first cable drive assembly operatively coupled to the anvil assembly via a first cable, the first cable disposed within the channel assembly;
a first trigger operatively coupled to the anvil assembly via the first cable, wherein an actuation of the first trigger causes the first cable to move from the protracted position to the retracted position;
a second cable drive assembly operatively coupled to the stapler assembly via a second cable, the second cable disposed within the channel assembly; and
a second trigger coupled to the stapler assembly via the second cable, wherein an actuation of the second trigger causes the stapler assembly to drive staples into a tissue.

2. The surgical instrument of claim 1, wherein the handle assembly further comprises:
an articulation control element; and
an articulation cable drive assembly coupled to the articulation control element and the tool assembly via an articulation cable.

3. The surgical instrument of claim 1, wherein the first cable drive assembly comprises a first gear spool comprising:
a first gear component configured to rotate upon an input from the first trigger; and
a first spool component operatively coupled to the first cable, wherein the first spool component is configured to move the first cable upon a rotation by the first gear component.

4. The surgical instrument of claim 3, further comprising a locking assembly comprising a locking lever, the locking lever comprising:
a pawl disposed within a housing of the handle assembly and biased against the first gear spool; and
a press point extending outside of the housing, wherein pressing the press point biases the pawl away from the first gear spool.

5. The surgical instrument of claim 3, wherein the second cable drive assembly comprises a second gear spool comprising:
a second gear component configured to rotate upon an input from the second trigger; and
a second spool component operatively coupled to the second cable, wherein the second spool component is configured to move the second cable upon a rotation by the second gear component.

6. The surgical instrument of claim 1, wherein the first cable drive assembly and the second cable drive assembly are operatively coupled, wherein a triggering of the first trigger causes the second trigger to articulate to a triggerable position.

7. The surgical instrument of claim 6, further comprising:
a first four-bar linkage coupled to the first trigger, comprising:
a first gear wedge configured to bias a first gear spool when the first trigger is pulled; and
an interlock pin disposed on the first gear wedge; and
a second four-bar linkage coupled to the second trigger, comprising:
a second gear wedge configured to bias a second gear spool when the second trigger is pulled; and
an interlock slot disposed on the second gear wedge configured to receive the interlock pin.

8. The surgical instrument of claim 1, wherein the first trigger comprises an inverted handle that pivots at a fulcrum.

9. The surgical instrument of claim 1, further comprising a pin interlock, wherein the pin interlock prevents the second trigger from being pulled until the first trigger is pulled.

10. The surgical instrument of claim 1, wherein the handle assembly is configured to trigger the tool assembly while the tool assembly is inserted into a subject via a trocar insertion site.

11. The surgical instrument of claim 1, wherein the surgical instrument is configured to staple tissues within a subject during a laparoscopic procedure.

12. A surgical instrument comprising:
a tool assembly;
a channel assembly coupled to the tool assembly on a distal end via an articulation joint;
a handle assembly coupled to the channel assembly on a proximal end, the handle assembly comprising:
a handle grip;
an articulation element; and
an articulation cable drive assembly coupled to the articulation element and the tool assembly via an articulation cable, wherein an adjustment of the articulation element causes the tool assembly to articulate greater than 60 degrees relative to the channel assembly.

13. The surgical instrument of claim 12, wherein the adjustment of the articulation element causes the tool assembly to articulate approximately 90 degrees relative to the channel assembly.

14. The surgical instrument of claim 12, wherein the articulation joint comprises a revolute articulation joint.

15. The surgical instrument of claim 12, wherein the surgical instrument includes a single revolute articulation joint.

16. The surgical instrument of claim 12, further comprising a first front idler pulley disposed within the articulation joint and a first rear idler pulley disposed within the tool assembly, wherein a first cable is threaded through the first front idler pulley and the first rear idler pulley.

17. The surgical instrument of claim 16, further comprising a second front idler pulley disposed within the articulation joint and a second rear idler pulley disposed within the tool assembly, wherein a second cable is threaded through the second front idler pulley and the second rear idler pulley.

18. The surgical instrument of claim 12, wherein the handle assembly is configured to trigger the tool assembly while the tool assembly is inserted into a subject via a trocar insertion site.

19. The surgical instrument of claim 12, wherein the surgical instrument is configured to staple tissues within a subject during a laparoscopic procedure.

20. A method for operating a surgical instrument comprising:
obtaining the surgical instrument, wherein the surgical instrument comprises:
a tool assembly comprising an anvil assembly and a stapler assembly;
a channel assembly coupled to the tool assembly on a distal end; and
a handle assembly coupled to the channel assembly on a proximal end comprising a first trigger and a second trigger;
inserting the tool assembly into a body cavity while the anvil assembly is in a clamped position relative to the stapler assembly;
opening the anvil assembly to an open position relative to the stapler assembly;
positioning tissue between the anvil assembly and the stapler assembly;
pulling a first trigger, wherein pulling the first trigger moves the anvil assembly into a clamped position, and moves the second trigger into a triggerable position; and
pulling a second trigger, wherein pulling the second trigger causes the stapler assembly to drive staples into the tissue.
